(19) United States
(12) Patent Application Publication
(10) Pub. No.: US 2021/0228128 A1
(43) Pub. Date: Jul. 29, 2021

(54) SENSING SYSTEMS AND METHODS FOR IDENTIFYING EMOTIONAL STRESS EVENTS

(71) Applicant: Abbott Diabetes Care Inc., Alameda, CA (US)

(72) Inventors: Benjamin J. Feldman, Berkeley, CA (US); Hyun Cho, Berkeley, CA (US); Meghan Sullivan Thompson, Livermore, CA (US)

(73) Assignee: Abbott Diabetes Care Inc., Alameda, CA (US)

(21) Appl. No.: 17/051,033

(22) PCT Filed: Mar. 6, 2019
(86) PCT No.: PCT/US19/20931

§ 371 Date: Oct. 27, 2020

Related U.S. Application Data
(60) Provisional application No. 62/668,348, filed on May 8, 2018.

(51) Int. Cl. A61B 5/16; A61B 5/1486; A61B 5/00; A61B 5/145; A61B 5/11; G16H 40/67; G16H 20/30

(52) U.S. Cl. CPC A61B 5/165; A61B 5/1486; A61B 5/6801; G16H 20/30; A61B 5/1118; A61B 5/7475; G16H 40/67; A61B 5/14546

(57) ABSTRACT

Changes in lactate concentrations in vivo may be indicative of a number of physiological conditions, among which can be emotional stress events. Sensing systems for emotional stress events may comprise a lactate-responsive sensor adapted for detecting lactate in vivo, and a processor communicatively coupled to the lactate-responsive sensor. The processor is adapted to determine a plurality of lactate concentrations measured by the lactate-responsive sensor over a period of time. The processor is further adapted to correlate a lactate concentration spike, a lactate concentration change, and/or a lactate concentration rate of change to an emotional stress event occurring within the period of time. The systems may exclude data received during designated or specified sleeping times, exercise, or after eating in order to determine a genuine emotional stress event.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND

The detection of various analytes within an individual can sometimes be vital for monitoring the condition of their health and well-being. Deviation from normal analyte levels can often be indicative of an underlying physiological condition, such as a metabolic condition or illness. Glucose levels, for example, can be particularly important to detect and monitor in diabetic individuals. Levels of certain analytes may also change episodically in response to various environmental factors or stimuli.

Lactate is one analyte whose in vivo levels may vary in response to numerous environmental or physiological factors including, for example, eating, stress, exercise, sepsis or septic shock, hypoxia, presence of cancerous tissue, and the like. In the case of chronic, ongoing conditions, such as sepsis, septic shock, or cancer, periodic laboratory measurements of lactate levels may be sufficient to determine whether these conditions are increasing or decreasing in severity. Periodic laboratory measurements of lactate levels may be wholly inadequate for monitoring short-lived, acute events, however. Namely, the measurement frequency may not be regular enough to observe a lactate spike (increase) above baseline in an individual experiencing a lactate-changing stimulus. Even if a lactate spike is observed when measuring lactate levels with periodic laboratory measurements, there is often is no possibility of taking proactive actions to alleviate or remediate a particular condition leading to the enhanced lactate levels. This can have significant consequences for a user's health and well-being in some cases.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 shows a diagram of an illustrative sensing system that may incorporate a lactate-responsive sensor of the present disclosure.

FIG. 2A shows a diagram of an illustrative two-electrode sensor configuration compatible for use in the disclosure herein.

FIG. 2B shows a diagram of an illustrative three-electrode sensor configuration compatible for use in the disclosure herein. FIG. 2C shows a diagram of another configuration of an illustrative three-electrode sensor configuration compatible for use in the disclosure herein.

FIG. 3 shows an illustrative decision tree that may be executed by a processor within the sensing systems of the present disclosure, in which elevated lactate levels occurring during sleep or exercise may be determined as being unrelated to an emotional stress event.

FIG. 4 shows an illustrative decision tree that may be executed by a processor within the sensing systems of the present disclosure, in which elevated lactate levels occurring during sleep or exercise or as a result of eating may be determined as being unrelated to an emotional stress event.

FIG. 5 shows a plot of in vivo measured lactate concentrations as a function of time.

### DETAILED DESCRIPTION

The present disclosure generally describes methods for monitoring and managing emotional stress and, more specifically, systems and methods for monitoring lactate in vivo for management of emotional stress.

As discussed above, lactate concentrations in an individual may be diagnostic of exposure to various physiological and environmental factors. Laboratory measurements of lactate concentrations may be sufficient to monitor chronic lactate-altering conditions, but such an approach may be wholly inadequate when acute factors or conditions alter lactate concentrations in vivo, especially when lactate concentrations fluctuate rapidly in response to a particular stimulus. More specifically, laboratory measurements of lactate concentrations may not occur with sufficient frequency or analysis rapidity to observe a lactate spike, and even if observed, it may not be possible to take proactive action in response to an irregular (elevated) lactate concentration. While some factors leading to elevated lactate concentrations are benign, other factors may lead to problematic health consequences if not addressed in a suitable manner.

In contrast to laboratory measurements of lactate concentrations, analyte sensors that are responsive to lactate in vivo may be operable to provide a plurality of lactate concentrations with sufficient frequency and analytical rapidity to observe lactate spikes during or shortly after their occurrence (i.e., in real-time or near real-time). As such, the analyte sensors described herein may facilitate a proactive response to lactate-altering physiological and/or environmental factors, should the factor be identified as substantially non-benign in nature and worthy of proactive management. Proactive management of emotional stress represents one instance where monitoring lactate concentrations in vivo may allow substantial health and well-being benefits to be realized.

Moreover, the present disclosure further describes sensing systems incorporating a lactate-responsive sensor, in which various sensing components, such as a processor and/or instructions coded therein, are adapted to identify various lactate-altering factors that may benignly cause a lactate spike or similar lactate concentration deviation. Example factors leading to a benign (harmless) lactate spike may include, for instance, exercise or eating. As such, the sensing systems described herein may be adapted to discriminate between lactate spikes arising from exposure to benign or non-benign lactate-altering factors. Thus, the sensing systems described herein may allow a user to proactively address elevated and/or rapidly changing lactate concentrations that are potentially related to various health and well-being issues. The sensing systems may further query a user (i.e., a primary user or wearer of a lactate-responsive sensor), a designated individual, or a group concerning the individual's health and/or emotional status in light of an observed lactate spike, which may further facilitate proactive health and well-being management, according to certain embodiments. Alternately, the sensing systems may query a user about their lifestyle during a given period of time, and the sensing systems may retrospectively analyze the sensor data to determine if a correlation exists between particular lifestyle events (e.g., those producing emotional stress) and an observed lactate spike or similar lactate concentration irregularity.

As such, the sensing systems described herein may advantageously help a user better manage emotional stress by maintaining lactate concentrations within a desired range. While emotional stress is an unavoidable consequence of daily life, the sensing systems described herein may help decrease the intensity of an emotional stress event by informing a user or other individual when lactate concentrations are indicative that a severe emotional stress event is occurring, has occurred, or is about to occur. Improved health and well-being outcomes therefore may result from proactive management of the emotional stress event.

Accordingly, sensing systems of the present disclosure may comprise: a lactate-responsive sensor adapted for detecting lactate in vivo; and a processor communicatively coupled to the lactate-responsive sensor. The processor is adapted to determine a plurality of lactate concentrations measured by the lactate-responsive sensor over a period of time. The processor is further adapted to correlate a lactate spike, a lactate concentration change, and/or a lactate concentration rate of change to an emotional stress event occurring within the period of time.

Emotional stress events that may be identified by the sensing systems and methods of the present disclosure include, but are not limited to, arguments, fights, relationship issues, emotional or physical trauma, terror, fear, paranoia, anxiety, and the like. Identification may occur through correlation to a lactate spike, lactate concentration change, and/or lactate concentration rate of change. Benign lactate-altering factors that may be identified as such by the sensing systems include, for example, exercise and eating. Thus, a lactate spike, lactate concentration change, and/or lactate concentration rate of change occurring in conjunction with a benign lactate-altering factor is not generally correlated with a genuine emotional stress event, such as those noted above. Additional lactate-altering factors that can be definitively determined as not arising from emotional stress (e.g., during sleep) may be identified by the sensing systems as well.

Additional details concerning illustrative lactate-responsive sensors suitable for incorporation within the sensing systems and methods of the present disclosure are provided hereinafter. It is to be appreciated, however, that lactate-responsive sensors having architectures, configurations, and/or components different than or in addition to those described expressly hereinafter may also be used suitably in some embodiments of the present disclosure. In general, any lactate-responsive sensor that is suitable for in vivo disposition may be used in the various embodiments of the present disclosure. In particular embodiments, a housing for the lactate-responsive sensor may be adapted to be worn on-body, and at least a portion of the lactate-responsive sensor may protrude from the housing for insertion in vivo. An active sensing region may be located upon at least a portion of the portion of the lactate-responsive sensor protruding from the housing, particularly a portion of the sensor configured for insertion in vivo. The active sensing region may comprise a sensing layer comprising a polymer and a lactate-responsive enzyme, according to various embodiments.

The active sensing region of lactate-responsive sensors of the present disclosure may be situated in any suitable location in vivo. Suitable locations may include, but are not limited to, intravenous, subcutaneous, or dermal locations. Intravenous sensors have the advantage of analyzing lactate directly in blood, but they are invasive and can sometimes be painful for an individual to wear over an extended period. Subcutaneous and dermal analyte sensors can often be less painful for an individual to wear and can provide sufficient measurement accuracy in many cases. In more particular embodiments, certain lactate-responsive sensors suitable for use in the present disclosure may be dermal sensors configured to assay dermal fluid of a user.

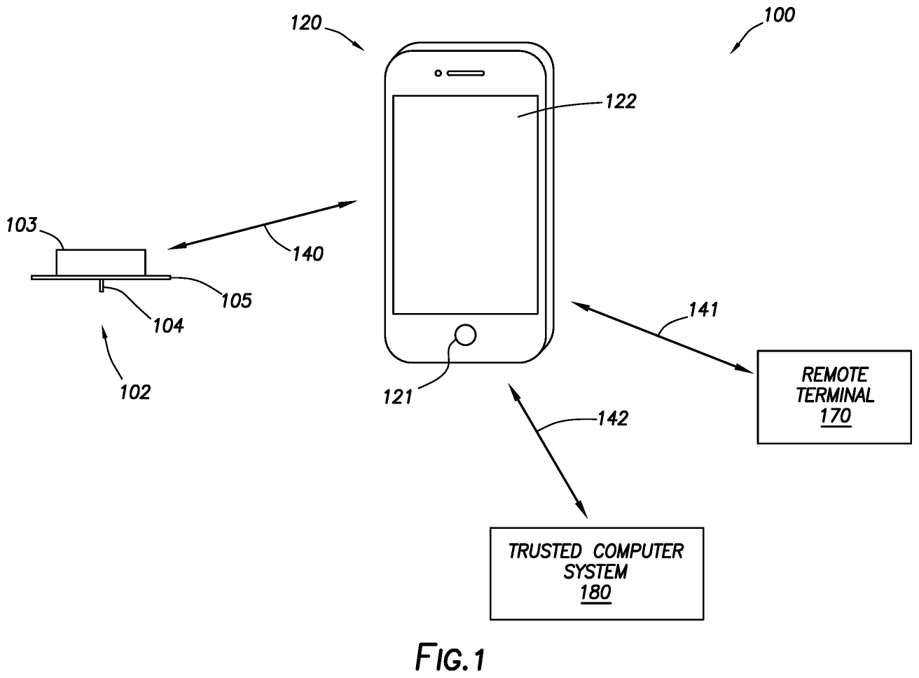

FIG. 1 shows a diagram of an illustrative sensing system that may incorporate a lactate-responsive sensor of the present disclosure. As shown, sensing system 100 includes sensor control device 102 and reader device 120 that are configured to communicate with one another over a local communication path or link, which may be wired or wireless, uni- or bi-directional, and encrypted or non-encrypted. Reader device 120 may constitute an output medium for viewing lactate concentrations and alerts or notifications determined by sensor 104 or a processor associated therewith, as well as allowing for one or more user inputs, according to some embodiments. Reader device 120 may also be in communication with remote terminal 170 and/or trusted computer system 180 via communication path(s)/link(s) 141 and/or 142, respectively, which also may be wired or wireless, uni- or bi-directional, and encrypted or non-encrypted. Any suitable electronic communication protocol may be used for each of the communication paths or links, such as near field communication (NFC), radio frequency identification (RFID), BLUETOOTH® or BLUETOOTH® Low Energy protocols, WiFi, or the like. Remote terminal 170 and/or trusted computer system 180 may be accessible, according to some embodiments, by individuals other than a primary user who have an interest in the user's lactate levels or emotional stress events (e.g., parents, siblings, physicians, therapists, teachers, and the like). Reader device 120 may comprise display 122 and optional input component 121. Display 122 may comprise a touch-screen interface, according to some embodiments.

Sensor control device 102 includes sensor housing 103, which may house circuitry and a power source for operating sensor 104. A processor (not shown) may be communicatively coupled to sensor 104, with the processor being physically located within sensor housing 103 or reader device 120. Sensor 104 protrudes from the underside of sensor housing 103 and extends through adhesive layer 105, which is adapted for adhering sensor housing 103 to a tissue surface, such as skin, according to some embodiments.

Sensor 104 is adapted to be at least partially inserted into a tissue of interest, such as within the dermal layer of the skin. Sensor 104 may comprise a sensor tail of sufficient length for insertion to a desired depth in a given tissue. The sensor tail may comprise a sensing region or sensing layer that is active for sensing lactate, and may comprise a lactate-responsive enzyme, according to one or more embodiments. The sensing region or sensing layer may include a polymeric material to which the lactate-responsive enzyme is covalently bonded, according to some embodiments. In various embodiments of the present disclosure, lactate may be monitored in any biological fluid of interest such as dermal fluid, plasma, blood, lymph, synovial fluid, cerebrospinal fluid, saliva, bronchoalveolar lavage, amniotic fluid, or the like. In particular embodiments, lactate-responsive sensors of the present disclosure may be adapted for assaying dermal fluid.

An introducer may be present transiently to promote introduction of sensor 104 into a tissue. In illustrative embodiments, the introducer may comprise a needle. It is to be recognized that other types of introducers, such as sheaths or blades, may be present in alternative embodiments. More specifically, the needle or similar introducer may transiently reside in proximity to sensor 104 prior to insertion and then be withdrawn afterward. While present, the needle or other introducer may facilitate insertion of sensor 104 into a tissue by opening an access pathway for sensor 104 to follow. For example, the needle may facilitate penetration of the epidermis as an access pathway to the dermis to allow implantation of sensor 104 to take place, according to one or more embodiments. After opening the access pathway, the needle or other introducer may be withdrawn so that it does not represent a sharps hazard. In illustrative embodiments, the needle may be solid or hollow, beveled or non-beveled, and/or circular or non-circular in cross-section. In more particular embodiments, the needle may be comparable in cross-sectional diameter and/or tip design to an acupuncture needle, which may have a cross-sectional diameter of about 250 microns. It is to be recognized, however, that suitable needles may have a larger or smaller cross-sectional diameter if needed for particular applications.

In some embodiments, a tip of the needle may be angled over the terminus of sensor 104, such that the needle penetrates a tissue first and opens an access pathway for sensor 104. In other illustrative embodiments, sensor 104 may reside within a lumen or groove of the needle, with the needle similarly opening an access pathway for sensor 104. In either case, the needle is subsequently withdrawn after facilitating insertion.

Sensor 104 may employ a two-electrode or a three-electrode detection motif, according to various embodiments of the present disclosure. Three-electrode motifs may comprise a working electrode, a counter electrode, and a reference electrode. Two-electrode motifs may comprise a working electrode and a second electrode, in which the second electrode functions as both a counter electrode and a reference electrode (i.e., a counter/reference electrode). In both two-electrode and three-electrode detection motifs, the sensing region or sensing layer of sensor 104 may be in contact with the working electrode. In various embodiments, the various electrodes may be at least partially stacked upon one another, as described in further detail hereinafter. In alternative embodiments, the various electrodes may be spaced apart from one another upon the insertion tail of sensor 104.

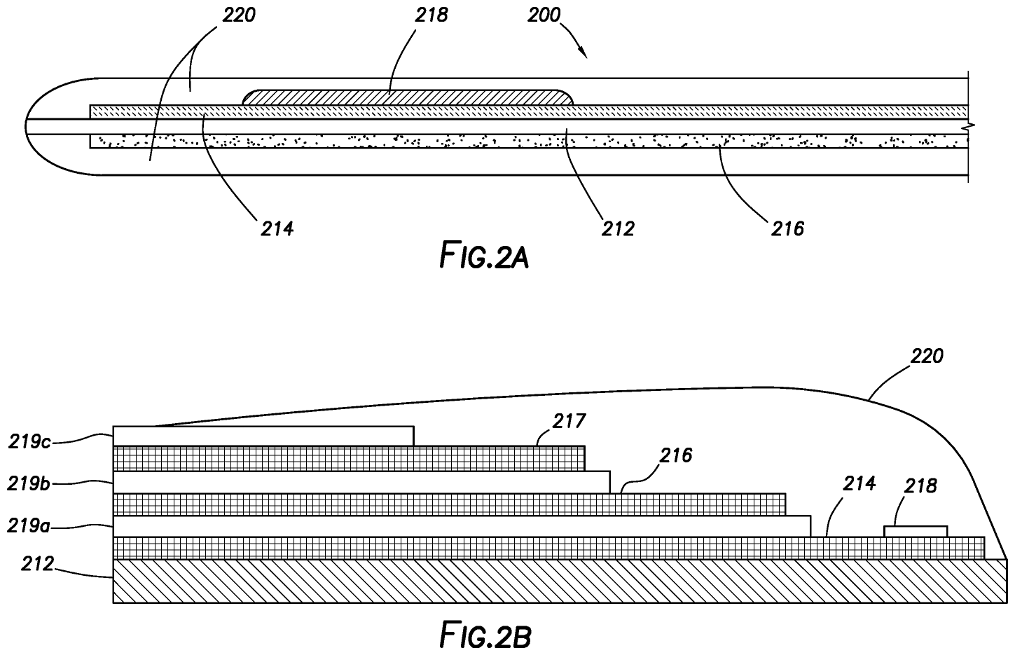

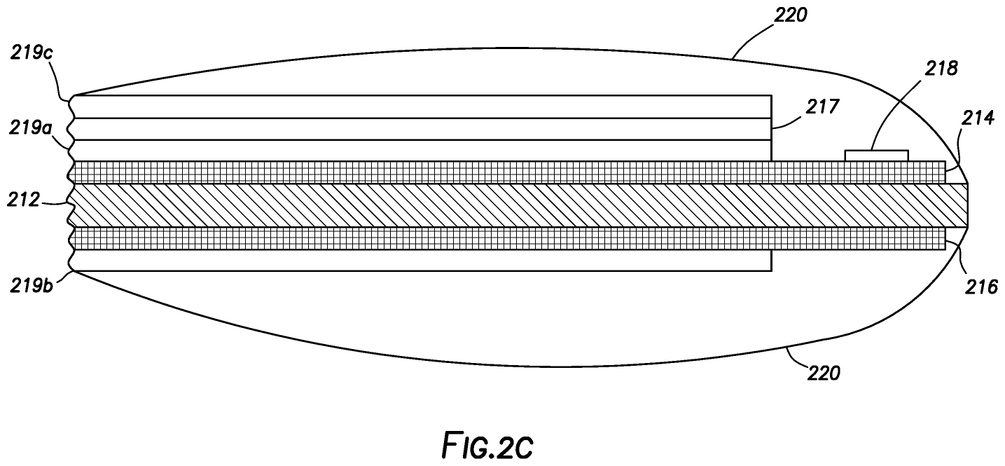

FIG. 2A shows a diagram of an illustrative two-electrode sensor configuration compatible for use in the disclosure herein. As shown, sensor 200 comprises substrate 212 disposed between working electrode 214 and counter/reference electrode 216. Alternately, working electrode 214 and counter/reference electrode 216 may be located upon the same side of substrate 212 with a dielectric material interposed in between. Sensing region 218 is disposed as at least one layer upon at least a portion of working electrode 214. In some embodiments, sensing region 218 may comprise multiple spots or a single spot configured for detection of an analyte of interest, such as lactate. Membrane 220 overcoats at least sensing region 218 and may optionally overcoat some or all of working electrode 214 and/or counter/reference electrode 216, in some embodiments. One or both faces of sensor 200 may be overcoated with membrane 220. While not shown, in some embodiments, membrane 220 may also cover the entirety of sensor 200, including substrate 212. Membrane 220 may comprise a polymer having capabilities of limiting analyte flux to sensing region 218, specifically the lactate flux in the disclosure herein. Sensor 200 may be operable for assaying lactate by any of coulometric, amperometric, voltammetric, or potentiometric electrochemical detection techniques.

Three-electrode sensor configurations may be similar to that shown for sensor 200, except for the inclusion of an additional electrode (FIGS. 2B and 2C). With additional electrode 217, counter/reference electrode 216 may then function as either a counter electrode or a reference electrode, and additional electrode 217 (FIGS. 2B and 2C) fulfills the other electrode function not otherwise fulfilled. Working electrode 214 continues to fulfill this function. The additional electrode 217 may be disposed upon either working electrode 214 or counter/reference electrode 216, with a separating layer of dielectric material in between. For example, as depicted in FIG. 2B dielectric layers 219a, 219b and 219c separate electrodes 214, 216 and 217 from one another. Alternately, at least one of electrodes 214, 216 and 217 may be located upon opposite faces of substrate 212 (FIG. 2C). Thus, in some embodiments, electrode 214 (working electrode) and electrode 216 (counter electrode) may be located upon opposite faces of substrate 212, with electrode 217 (reference electrode) being located upon one of electrodes 214 or 216 and spaced apart therefrom with a dielectric material. As with sensor 200 shown in FIG. 2A, sensing region 218 may comprise multiple spots or a single spot configured for detection of an analyte of interest, such as lactate.

Additional electrode 217 may be overcoated with membrane 220 in some embodiments. Although FIGS. 2B and 2C have depicted all of electrodes 214, 216 and 217 as being overcoated with membrane 220, it is to be recognized that only working electrode 214 may be overcoated in some embodiments. Moreover, the thickness of membrane 220 at each of electrodes 214, 216 and 217 may be the same or different. As such, the configuration shown in FIGS. 2B and 2C should be understood as being non-limiting of the embodiments disclosed herein. As in two-electrode configurations, one or both faces of sensor 200 may be overcoated with membrane 220.

In some embodiments, sensing region 218 may comprise a lactate-responsive enzyme. More particularly, the lactate-responsive enzyme may comprise lactate dehydrogenase or lactate oxidase, according to various embodiments. In some embodiments, sensing region 218 may further comprise a stabilizer for lactate dehydrogenase or lactate oxidase, such as catalase. According to still more specific embodiments, the lactate-responsive enzyme, such as lactate dehydrogenase or lactate oxidase, may be covalently bonded to a polymer comprising sensing region or sensing layer 218. Covalent bonding immobilizes the lactate-responsive enzyme upon working electrode 214.

In still more specific embodiments, sensing region or sensing layer 218 may comprise a polymer that is covalently bonded to the lactate-responsive enzyme, such as lactate dehydrogenase or lactate oxidase, and a low-potential osmium complex electron transfer mediator, as disclosed in, for example, U.S. Pat. No. 6,134,461, which is incorporated herein by reference in its entirety. The electron transfer mediator may facilitate conveyance of electrons from lactate to working electrode 214 during a redox reaction. Changes in the signal intensity (e.g., current) at working electrode 214 may be proportional to the lactate concentration and/or the activity of the lactate-responsive enzyme. A calibration factor may be applied to determine the lactate concentration from the signal intensity, according to some embodiments. Suitable electron transfer mediators include electroreducible and electrooxidizable ions, complexes or molecules having redox potentials that are a few hundred millivolts above or below the redox potential of the standard calomel electrode (SCE). Other suitable electron transfer mediators may comprise metal compounds or complexes of ruthenium, iron (e.g., polyvinylferrocene), or cobalt, for example. Suitable ligands for the metal complexes may include, for example, bidentate or higher denticity ligands such as, for example, a bipyridine, biimidazole, pheanthroline, or pyridyl(imidazole). Other suitable bidentate ligands may include, for example, amino acids, oxalic acid, acetylacetone, diaminoalkanes, or o-diaminoarenes. Any combination of monodentate, bidentate, tridentate, tetradentate, or higher denticity ligands may be present in the metal complex to achieve a full coordination sphere.

Suitable polymers for inclusion in sensing region 218 include, but are not limited to, polyvinylpyridines (e.g., poly(4-vinylpyridine)), polyimidazoles (e.g., poly(l-vinylimidazole), or any copolymer thereof. Illustrative copolymers that may be suitable include, for example, copolymers containing monomer units such as styrene, acrylamide, methacrylamide, or acrylonitrile.

Covalent bonding of the lactate-responsive enzyme to a polymer or other matrix (e.g., sol-gel) in sensing region 218 may take place via a crosslinker introduced with a suitable crosslinking agent. Suitable crosslinking agents for reaction with free amino groups in the enzyme (e.g., with the free amine in lysine) may include crosslinking agents such as, for example, polyethylene glycol diglycidylether (PEGDGE) or other polyepoxides, cyanuric chloride, N-hydroxysuccinimide, imidoesters, epichlorohydrin, or derivatized variants thereof. Suitable crosslinking agents for reaction with free carboxylic acid groups in the enzyme may include, for example, carbodiimides.

Although the lactate-responsive enzyme and/or the electron transfer mediator may be covalently bonded to a polymer or other suitable matrix in sensing region 218, other association means may be suitable as well. In some embodiments, the lactate-responsive enzyme and/or the electron transfer mediator may be ionically or coordinatively associated with the polymer or other matrix. For example, a charged polymer may be ionically associated with an oppositely charged lactate-responsive enzyme or electron transfer mediator. In still other embodiments, the lactate-responsive enzyme and/or the electron transfer mediator may be physically entrained within the polymer or other matrix of sensing region 218.

In alternative embodiments, one or more components of sensing region 218 may be solvated, dispersed, or suspended in a fluid, instead of being disposed in a solid composition. The fluid may be provided with sensor 200 or may be absorbed by sensor 200 from the biological fluid that is undergoing analysis. In some embodiments, the components which are solvated, dispersed, or suspended in this type of sensing region 218 are non-leachable from sensing region 218. In some embodiments, non-leachability may be accomplished, for example, by providing barriers (e.g., membranes and/or films) around sensing region 218. One example of such a barrier is a microporous membrane or film, which allows diffusion of lactate into sensing region 218, but reduces or eliminates diffusion of sensing region 218 components (e.g., an electron transfer agent, an enzyme and/or a reactant) out of sensing region 218. Such barriers may, in some embodiments, be considered as flux-limiting membranes and may avoid saturating sensor 200 when excessive lactate is present. Flux-limiting membranes of this type may also be used when sensing region contains primarily solid components, as referenced above.

Sensor 200 may also be configured to analyze for other analytes as well. For example, according to some embodiments, sensor 200 may be further adapted for detecting glucose in vivo by also incorporating suitable sensing functionality for this analyte.

It is to be appreciated that analyte monitoring system 100 and sensor 200 may comprise additional features and/or functionality that are not necessarily described herein in the interest of brevity. Thus, the foregoing description of analyte monitoring system 100 and sensor 200 should be considered illustrative and non-limiting in nature.

Accordingly, methods of the present disclosure may comprise: assaying a biological fluid in vivo with a lactate-responsive sensor adapted for measuring lactate in the biological fluid over a period of time; communicating a signal from the lactate-responsive sensor to a processor; determining a plurality of lactate concentrations with the processor using the signal communicated from the lactate-responsive sensor; correlating, with the processor, a lactate concentration spike, a lactate concentration change, a lactate concentration rate of change, or any combination thereof to an emotional stress event occurring within the period of time; and communicating a notification from the processor to an output medium that an emotional stress event has occurred. Additional method details are provided hereinbelow.

As referenced above, the sensing systems of the present disclosure may be adapted to determine whether elevated or changing lactate concentrations may be correlated with an emotional stress event. More specifically, the processor in communication with the lactate-responsive sensor may be adapted to correlate a lactate concentration spike, a lactate concentration change, a lactate concentration rate of change, or any combination thereof to an emotional stress event occurring within a given period of time. That is, the processor may analyze and identify lactate concentrations above a specified threshold value, a change in lactate concentrations (regardless of the absolute magnitude of the lactate concentrations themselves), or the rate at which the lactate concentrations are changing. Analysis of the rate of change may be especially useful to help identify potential emotional stress events as they occur by identifying concentration trends indicating that a specified lactate concentration threshold may be exceeded. Doing so may facilitate a more rapid response to an emotional stress event. In some embodiments, the specified threshold may be determined based upon retrospective analysis of a given user's baseline lactate levels or those typically produced in an emotional stress event. In other embodiments, a threshold lactate level, such as about 1.5 mM or above, may be specified. Other suitable threshold lactate levels may include about 0.5 mM or above, or about 1 mM or above, or about 1.25 mM or above, or about 1.75 mM or above, or about 2.5 mM or above, for example. The chosen threshold level may depend, at least in part, upon the type of emotional stress that one intends to identify as well as to accommodate lactate concentration variability in different individuals. If one or more of the foregoing conditions are met, the processor may perform an additional query to determine if the elevated lactate levels can be correlated to a benign factor leading to elevated lactate levels, such as eating or exercising. Otherwise, the elevated lactate levels may be identified as being associated with an emotional stress event or potential emotional stress event. Once a potential emotional stress event has been identified, the sensing systems may perform additional actions or be adapted to perform additional actions, as described further hereinbelow.

The processor associated with the sensing systems is further adapted to correlate a measured lactate concentration, concentration change, and/or rate of concentration change with potential emotional stress-related events. In order to do so, the sensing systems may be adapted to identify other lactate-altering factors and/or determine if the elevated or changing lactate concentrations are unlikely to be associated with an emotional stress event. More specifically, the sensing systems may be adapted to determine whether other lactate-altering factors have occurred and if they occurred at the same time as the lactate spike, concentration change, or concentration rate of change.

Accordingly, in further embodiments, the sensing systems of the present disclosure may further comprise a clock, an actigraph (e.g., to track sleep times or sleep patterns), an exercise monitor (e.g., incorporating a pedometer, altimeter, temperature monitor, heart rate monitor, position monitor, accelerometer, or the like), or any combination thereof communicatively coupled to the processor. The processor is further adapted to determine, based upon a reading from the clock, the actigraph, the exercise monitor, or any combination thereof, that the lactate concentration spike, the lactate concentration change, the lactate concentration rate of change, or any combination thereof is not associated with an emotional stress event occurring within the period of time. Further details are provided below regarding how the processor utilizes data provided by a clock (time), actigraph (sleep patterns), and/or an exercise monitor (e.g., heart rate, position, acceleration or the like) to determine whether an emotional stress event has occurred.

In more specific embodiments, the sensing systems of the present disclosure may comprise a clock and an exercise monitor communicatively coupled to the processor, and optionally an actigraph. The processor is further adapted to determine, based upon a reading from the clock or actigraph and a reading from the exercise monitor that the lactate concentration spike, the lactate concentration change, the lactate concentration rate of change, or any combination thereof is not associated with an emotional stress event occurring within the period of time.

In still further embodiments, the sensing systems disclosed herein may further comprise a user input interface communicatively coupled to the processor. The user input interface is adapted such that a wearer (i.e., a primary user) may enter times associated with one or more of exercising, eating or sleeping. Thus, the sensing systems may further accept user input to aid in determining whether a lactate concentration spike, concentration change, and/or concentration rate of change is associated with an emotional stress event. The user inputs may override a time, sleep determination and/or exercise reading conveyed to the processor from a clock or exercise monitor. It is to be appreciated that a non-wearer, such as a parent, caregiver or other concerned individual, may additionally provide user input as well (e.g., if the primary user is unable to provide input or requires supervision). The user inputs may also be entered using a remote device or remote terminal, particularly when the individual providing the user inputs is not the primary user.

In some embodiments, the processor may be configured to determine if a lactate spike, concentration change, and/or concentration rate of change occurred at a time when a wearer (i.e., a primary user) of the sensor was likely to be sleeping. Since sleep is a time of relaxation and a wearer of the sensor is unlikely to experience a detrimental emotional stress event during sleep, the sensing system may be adapted to not report irregular or elevated lactate levels that occur when the wearer is sleeping. The lactate measurements obtained during sleep may still be recorded and/or archived, however. As such, the sensing systems may comprise a clock communicatively coupled to the processor, wherein the processor is configured to read the time specified by the clock and determine if the wearer is likely to be sleeping. The time identified by the processor as being designated or specified for sleep may be adjusted based upon individualized sleep schedules and other factors (e.g., input by a primary user or other individual), or may be automatic based on actimetry measurements that track movement of the wearer during sleep. In alternative embodiments, an actimeter may be read directly to determine if a wearer is sleeping when elevated or irregular lactate levels occur.

In some embodiments, the sensing systems may further query a wearer in response to irregular or elevated lactate levels that occur during a time designated or specified for sleeping. For example, the sensing systems may query whether the wearer was awake during the designated or specified sleep time, experienced a nightmare while sleeping, or watched a scary late night movie. Actimetry measurements may also be used alone or in combination with a clock to determine if the user was sleeping during a time when elevated or irregular lactate levels occurred. Upon confirming that a lactate spike, concentration change, or concentration rate of change occurred during sleep or due to a nightmare, the sensing systems may determine that a potentially harmful emotional stress event did not occur. However, should the wearer inform the sensing systems that they were indeed awake during the designated or specified sleep time or the sensing systems automatically find that the wearer is awake, the sensing systems may determine that an emotional stress event occurred. Thus, in certain instances, user input may override the designated sleep time to aid in identifying an emotional stress event. Should the sensing systems determine that the wearer is indeed awake (e.g., automatically using an actimetry reading), the sensing systems may query or prompt the wearer about their emotional state or suggest a suitable calming intervention. If the sensing systems cannot conclusively determine that the wearer is awake, no query or prompt is immediately made, so as to avoid unnecessarily waking the wearer.

As one of ordinary skill in the art will appreciate, elevated lactate levels may occur during exercise as a result of anaerobic metabolism. In some embodiments, the processor of the sensing systems may be adapted to determine if a lactate spike, concentration change, and/or concentration rate of change occurred at a time when a wearer of the sensor was exercising. An exercise monitor may be communicatively coupled with the processor in order to determine whether a wearer of the sensor was exercising when a lactate spike, concentration change, and/or concentration rate of change occurred. The exercise monitor, like the clock or the actigraph, may be integral to the lactate-responsive sensors or sensing systems of the present disclosure or provided as a separate component in electronic communication with the sensing systems. As such, the sensing systems may query data received from the exercise monitor and determine if a marker of exercise occurred during the time a lactate spike, concentration change, and/or concentration rate of change occurred. Markers of exercise may include, for example, an elevated heart rate, a lowered blood oxygen content, a change in position (e.g., a change in altitude, latitude/longitude, and/or step count increase, or the like), a change in acceleration, or any combination thereof. In some embodiments, the exercise monitor may be a designated fitness device, such as a Fitbit, Garmin, or a simple pulse monitoring device. In other embodiments, the processor may be in communication with a smartphone or smartwatch application designated for monitoring a user's fitness level. The exercise monitor may contain an internal clock, such that the exercise monitor can communicate the time exercise occurred to the processor. Alternately, a clock distinct from the exercise monitor may be synched with the exercise monitor and used to determine whether exercise was occurring during a designated time. The clock used for determining the time when exercise occurred may be the same clock as that used to determine whether a wearer of the sensor is likely to be sleeping. Alternately, the exercise monitor may include a separate clock as a component thereof.

Exercise monitors that may be communicatively coupled to the processor are not believed to be particularly limited. Illustrative exercise monitors that may be suitably in communication with the processor include, but are not limited to, a heart rate monitor, a position monitor (e.g., a GPS device or altimeter), an accelerometer, a pedometer, or any combination thereof.

In some or other embodiments, the sensing systems may further query a wearer whether they indeed exercised during a specified period of time. For example, the sensing system may query the wearer in response to a lactate concentration spike, concentration change, and/or concentration rate of change. An exercise query may also occur if a wearer is not wearing an exercise monitor at all or data from the exercise monitor is not properly communicated to the processor. In other embodiments, an exercise query may occur when the processor is unable to determine upon reading data from the exercise monitor whether an exercise event occurred. As such, if the wearer (i.e., a primary user) informs the sensing systems that exercise occurred during a designated period of time, the sensing systems may designate a lactate concentration spike, concentration change, and/or concentration rate of change as not being related to an emotional stress event.

In some or other embodiments, the sensing systems may query a wearer whether data recorded by the exercise monitor was indeed due to an exercise event. For example, an elevated heart rate measured by a pulse monitor might be incorrectly designated as an exercise event when, in fact, the elevated heart rate was due to an emotional stress event, such as an argument. Thus, in some embodiments, the sensing systems may override the identification of an exercise event and correlate the observed lactate concentration spike, concentration change, and/or concentration rate of change with an emotional stress event. The user of the sensing systems or another individual may override such identification.

In still other embodiments, the processor may query multiple types of exercise data received from the exercise monitor to determine whether an exercise event occurred. For example, an elevated heart rate occurring without an associated change in position, acceleration or elevation may not be designated as an exercise event, according to some embodiments. As such, if the exercise data is inconsistent, the processor may further query a wearer to determine whether an exercise event indeed occurred and/or designate an associated lactate concentration spike, concentration change, and/or concentration rate of change as being due to an emotional stress event.

Accordingly, methods of the present disclosure may further comprise: communicating one or more variables to the processor within the period of time, and determining with the processor based upon the one or more variables whether the lactate concentration spike, the lactate concentration change, and/or the lactate concentration rate of change is associated with an emotional stress event. The one or more variables may include, for example, time of day, acceleration, position, altitude, heart rate, step count, an actimetry reading, or any combination thereof.

In further embodiments, if the one or more variables are measured at a time of day specified for sleeping (or the user is determined to be sleeping using an associated actimeter), the lactate concentration spike, the lactate concentration change, and/or the lactate concentration rate of change is not communicated to the output medium as an emotional stress event. In still further embodiments, as discussed in more detail above, user input may override the designation of a time of day specified for sleeping. In some or other embodiments, methods of the present disclosure may comprise determining with the processor whether a user of the lactate-responsive sensor has been sleeping, and if the processor determines the user has been sleeping (either from user input or from time of day analysis or actimeter data), the lactate concentration spike, the lactate concentration change, and/or the lactate concentration rate of change is not communicated to the output medium as an emotional stress event, although the lactate measurement may still be stored in a memory of the sensing systems for a period of time, however.

In some or other further embodiments, if the one or more variables are determined by the processor to be suggestive of exercise, the lactate concentration spike, the lactate concentration change, and/or the lactate concentration rate of change is not communicated to the output medium as an emotional stress event. In still further embodiments, as discussed in more detail above, user input may override the designation of an exercise event at a given time. In some or other embodiments, methods of the present disclosure may comprise determining with the processor whether a user of the lactate-responsive sensor has been exercising, and if the processor determines the user has been exercising (either from user input or analysis of data from an exercise monitor), the lactate concentration spike, the lactate concentration change, and/or the lactate concentration rate of change is not communicated to the output medium as an emotional stress event, although the lactate measurement may still be stored in a memory of the sensing systems for a period of time, however.

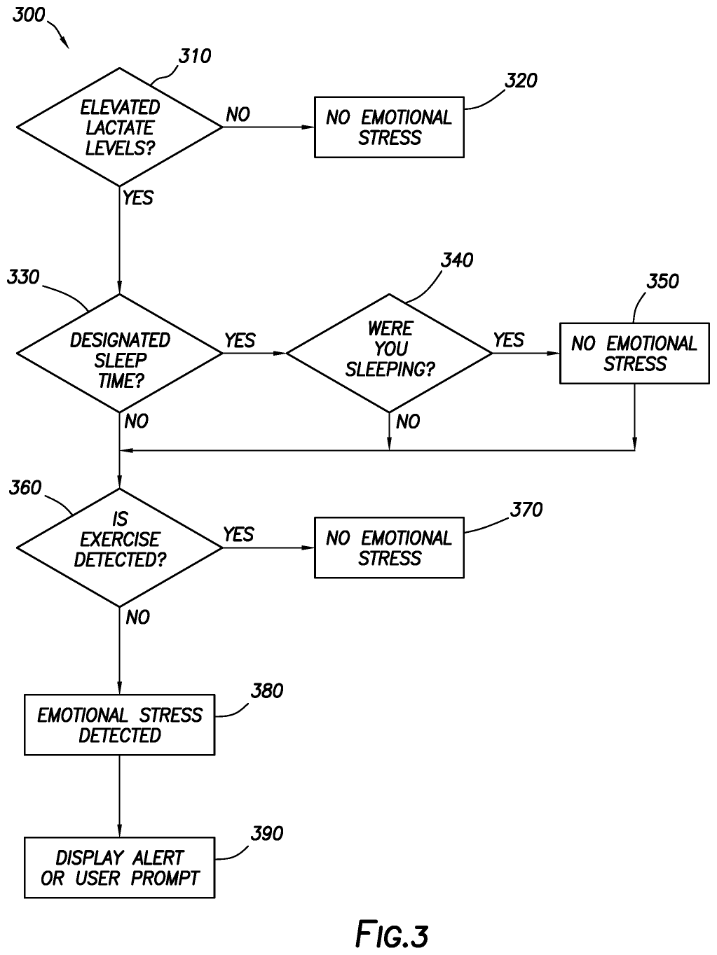

FIG. 3 shows an illustrative decision tree that may be executed by the processor within the sensing systems of the present disclosure, in which elevated lactate levels occurring during sleep or exercise may be determined as being unrelated to an emotional stress event. It is to be recognized that the decision order shown in FIG. 3 need not necessarily occur in the order depicted.

As shown in decision tree 300, elevated or irregular lactate concentrations may be detected in operation 310. If the lactate spike, change in lactate concentration, and/or rate of change in lactate concentration does not exceed one or more threshold values, emotional stress is not determined in operation 320. If one or more threshold values are exceeded in operation 310, decision tree 300 next evaluates in operation 330 whether a clock indicates a time designated for sleeping or an actimeter indicates that a user is possibly sleeping based on the user's level of activity or movement. As used herein, the term “designated sleep time” encompasses any of an automated determination of sleep through actimetry and/or from time of day analysis with a clock. If the clock or an actimeter indicates a designated sleep time, decision tree 300 may optionally query in operation 340 whether a user was indeed sleeping. If so, operation 350 may determine that an emotional stress event has not occurred. Otherwise, decision tree 300 may continue evaluating in operation 360 whether other factors may be affecting the lactate concentration. Namely, operation 360 may determine whether a user has been exercising. If operation 360 determines that a user has been exercising, operation 370 determines that an emotional stress event has not occurred. If no exercise is detected by operation 360 in decision tree 300, operation 380 may then determine that an emotional stress event has occurred. An alert or user prompt may be suggested in operation 390, according to some embodiments.

Lactate concentrations may also fluctuate as a consequence of eating. As such, the sensing systems of the present disclosure may further query a user whether they ate during or prior to the time when a lactate spike, change in lactate concentration, and/or rate of change in lactate concentration occurred. Namely, according to various embodiments, the user may enter a time when they ate, or the user may respond to a query from the processor whether they ate at a specified time (i.e., a time during or before that at which a lactate spike, change in lactate concentration, and/or rate of change in lactate concentration was observed).

Accordingly, methods of the present disclosure may further comprise: determining with the processor whether a user (wearer) of the lactate-responsive sensor has been eating. Determining may comprise the user (wearer) responding to one or more queries from the processor to evaluate whether eating occurred at a specified time. In addition to the wearer of the lactate-responsive sensor, other individuals may respond to an eating query according to various embodiments.

In other various embodiments, the sensing systems may evaluate blood glucose levels in vivo in order to evaluate whether a user has recently eaten. If blood glucose levels are rising, for example, the sensing systems may infer that a user has recently consumed food. Sensing of this type may be overridden if the user experiences irregular blood glucose levels, such as in diabetic individuals. A sensor separate from the lactate-responsive sensor may be used for evaluating blood glucose levels in vivo, with information from the separate sensor being communicated to the sensing systems. If the processor determines the user has been eating, the lactate spike, change in lactate concentration, and/or rate of change in lactate concentration is not communicated to the output medium as an emotional stress event. As such, the sensing systems of the present disclosure may further comprise a glucose-responsive sensor, according to various embodiments. The glucose-responsive sensor may comprise a processor that is communicatively coupled with the processor associated with the lactate-responsive sensor. In alternative embodiments, the processor associated with the lactate-responsive sensor may be further adapted to determine glucose concentrations measured by the glucose-responsive sensor

In still more specific embodiments, methods of the present disclosure may comprise: determining with the processor whether a user (wearer) of the lactate-responsive sensor has been sleeping, exercising, eating, or any combination thereof. If the processor determines the user (wearer) has been sleeping, exercising, eating, or any combination thereof, the lactate spike, the change in lactate concentration, and/or the rate of change in lactate concentration is not communicated to the output medium as an emotional stress event.

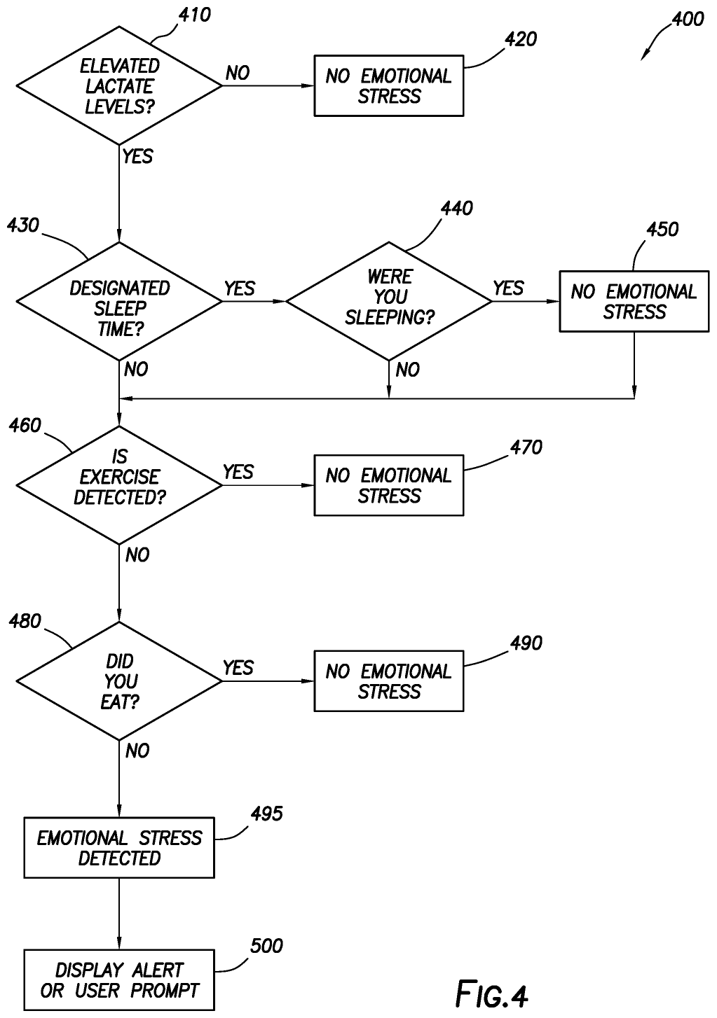

FIG. 4 shows an illustrative decision tree that may be executed by the processor within the sensing systems of the present disclosure, in which elevated lactate levels occurring during sleep or exercise or as a result of eating may be determined as being unrelated to an emotional stress event. It is likewise to be recognized that the decision order shown in FIG. 4 need not necessarily occur in the order depicted.

Decision tree 400 of FIG. 4 is similar in several aspects to decision tree 300 of FIG. 3. Namely, operations 400, 410, 420, 430, 440, 450, 460, and 470 in decision tree 400 substantially correspond to operations 300, 310, 320, 330, 340, 350, 360, and 370 in decision tree 300 and are not described again in the interest of brevity. Continuing with decision tree 400, if operation 460 determines that a user did not exercise during a specified time, operation 480 may then query or determine (e.g., by evaluating blood glucose concentrations) whether a user ate recently or at a specified time. If the user ate during a specified time period, operation 490 may determine that an emotional stress event has not occurred. In contrast, if operation 480 determines that eating did not occur, operation 495 may then communicate to an output medium that an emotional stress event may have occurred. An alert or user prompt may be suggested in operation 500, according to some embodiments.

Optionally, the sensing systems and methods of the present disclosure may be further adapted to interface or interact with a user (wearer) to notify and/or intervene with the user in the case an emotional stress event being detected (e.g., operations 390 and 500 in FIGS. 3 and 4). In some embodiments, the sensing systems and methods may suggest further action on the part of a user in the event of emotional stress being detected. Interfacing and/or interacting with a user may take place in any of a variety of forms or actions. In some embodiments, the sensing systems and methods may query a user concerning their emotional state (e.g., “Are you okay?”). In some embodiments, the sensing systems and methods may suggest performing a relaxation technique, taking medication, or the like. In some embodiments, the sensing systems and methods may convey an audible, visual, or other sensory-stimulating alarm (e.g., a haptic or mild electric shock) to a user should an emotional stress event be detected.

Accordingly, the methods of the present disclosure may further comprise prompting a user of the lactate-responsive sensor for a response after communicating a notification of an emotional stress event. In some or other embodiments, methods of the present disclosure may further comprise receiving one or more user inputs with the processors, and determining with the processor, based upon the one or more user inputs, whether the lactate concentration spike, the lactate concentration change, and/or the lactate concentration rate of change is associated with an emotional stress event. In some embodiments, the one or more user inputs may comprise a range of times associated with one or more of exercising, eating, sleeping, or any combination thereof. In some or other embodiments, the one or more user inputs may comprise a response to a query from the sensing systems, such as the user's emotional state or whether the user has taken action to relieve emotional stress.

In some or other embodiments, the sensing systems and methods may relay a notification to another person designated by the user (wearer), who may intervene on the user's behalf. The notification to the other person may occur in addition to a notification provided to the user, or as an alternative to a notification provided to a user. For example, the sensing systems and methods may be adapted to relay a notification to a user's spouse, parent, child, teacher, friend, doctor, therapist, or other individual who may interface with the user and potentially help them in relieving their emotional stress, particularly if the user is unable to perform such actions on their own.

Embodiments disclosed herein include:

A. Sensing systems for determining emotional stress events. The sensing systems comprise: a lactate-responsive sensor adapted for detecting lactate in vivo; and a processor communicatively coupled to the lactate-responsive sensor; wherein the processor is adapted to determine a plurality of lactate concentrations measured by the lactate-responsive sensor over a period of time, and wherein the processor is further adapted to correlate a lactate concentration spike, a lactate concentration change, a lactate concentration rate of change, or any combination thereof to an emotional stress event occurring within the period of time.

B. Methods for monitoring emotional stress. The methods comprise: assaying a biological fluid in vivo with a lactate-responsive sensor adapted for measuring lactate in the biological fluid over a period of time; communicating a signal from the lactate-responsive sensor to a processor; determining a plurality of lactate concentrations with the processor using the signal communicated from the lactate-responsive sensor; correlating, with the processor, a lactate concentration spike, a lactate concentration change, a lactate concentration rate of change, or any combination thereof to an emotional stress event occurring within the period of time; and communicating a notification from the processor to an output medium that an emotional stress event has occurred.

Each of embodiments A and B may have one or more of the following additional elements in any combination

Element 1: wherein a housing for the lactate-responsive sensor is adapted to be worn on-body, and at least a portion of the lactate-responsive sensor protrudes from the housing for insertion in vivo.

Element 2: wherein the lactate-responsive sensor comprises a working electrode having a sensing layer comprising a lactate-responsive enzyme disposed thereupon.

Element 3: wherein the lactate-responsive enzyme is covalently bonded to a polymer comprising the sensing layer.

Element 4: wherein the sensing layer further comprises catalase as a stabilizer for the lactate-responsive enzyme.

Element 5: wherein the lactate-responsive enzyme is lactate dehydrogenase or lactate oxidase.

Element 6: wherein the sensing system further comprises: a clock, an actimeter, an exercise monitor, or any combination thereof communicatively coupled to the processor; wherein the processor is further adapted to determine, based upon a reading from the clock, the actimeter, the exercise monitor, or any combination thereof, that the lactate concentration spike, the lactate concentration change, the lactate concentration rate of change, or any combination thereof is not associated with an emotional stress event occurring within the period of time.

Element 7: wherein the sensing system further comprises: a clock and an exercise monitor communicatively coupled to the processor; wherein the processor is further adapted to determine, based upon a reading from the clock and a reading from the exercise monitor, that the lactate concentration spike, the lactate concentration change, the lactate concentration rate of change, or any combination thereof is not associated with an emotional stress event occurring within the period of time.

Element 8: wherein the sensing system further comprises: a user input interface communicatively coupled with the processor; wherein the user input interface is adapted for entering times associated with one or more of exercising, eating or sleeping.

Element 9: wherein the method further comprises: communicating one or more variables to the processor within the period of time, the one or more variables being selected from the group consisting of time of day, an actimetry reading, acceleration, position, altitude, heart rate, and any combination thereof; and determining with the processor based upon the one or more variables whether the lactate concentration spike, the lactate concentration change, the lactate concentration rate of change, or any combination thereof is associated with an emotional stress event.

Element 10: wherein, if the one or more variables are measured at a time of day specified for sleeping, the lactate concentration spike, the lactate concentration change, the lactate concentration rate of change, or any combination thereof is not communicated to the output medium as an emotional stress event.

Element 11: wherein, if the one or more variables are determined by the processor to be suggestive of exercise, the lactate concentration spike, the lactate concentration change, the lactate concentration rate of change, or any combination thereof is not communicated to the output medium as an emotional stress event.

Element 12: wherein the method further comprises: determining with the processor whether a user of the lactate-responsive sensor has been sleeping; wherein, if the processor determines the user has been sleeping, the lactate concentration spike, the lactate concentration change, the lactate concentration rate of change, or any combination thereof is not communicated to the output medium as an emotional stress event.

Element 13: wherein the method further comprises: determining with the processor whether a user of the lactate-responsive sensor has been exercising; wherein, if the processor determines the user has been exercising, the lactate concentration spike, the lactate concentration change, the lactate concentration rate of change, or any combination thereof is not communicated to the output medium as an emotional stress event.

Element 14: wherein the method further comprises: determining with the processor whether a user of the lactate-responsive sensor has been eating; wherein, if the processor determines the user has been eating, the lactate concentration spike, the lactate concentration change, the lactate concentration rate of change, or any combination thereof is not communicated to the output medium as an emotional stress event.

Element 15: wherein the method further comprises: determining with the processor whether a user of the lactate-responsive sensor has been sleeping, exercising, eating, or any combination thereof;

wherein, if the processor determines the user has been sleeping, exercising, eating, or any combination thereof, the lactate concentration spike, the lactate concentration change, the lactate concentration rate of change, or any combination thereof is not communicated to the output medium as an emotional stress event.

Element 16: wherein the method further comprises: prompting a user of the lactate-responsive sensor for a response after communicating the notification of the emotional stress event to the output medium.

Element 17: wherein the method further comprises: receiving one or more user inputs with the processor, the one or more user inputs comprising a range of times associated with one or more of exercising, eating, sleeping or any combination thereof; and determining with the processor based upon the one or more user inputs whether the lactate concentration spike, the lactate concentration change, the lactate concentration rate of change, or any combination thereof is associated with an emotional stress event.

By way of non-limiting example, exemplary combinations applicable to A and B include:

The sensing system of A in combination with elements 1 and 2; 1 and 3; 1, 3 and 4; 1 and 5; 1, 2 and 3; 1-4; 1, 2 and 5; 2 and 3; 2-4; 2 and 5; 2-5; 1 and 6; 2 and 6; 3 and 6; 5 and 6; 1 and 7; 2 and 7; 3 and 7; 5 and 7; 1 and 8; 2 and 8; 3 and 8; 4 and 8; 6 and 8; and 7 and 8. The method of B in combination with elements 1 and 2; 1 and 3; 1, 3 and 4; 1 and 5; 1, 2 and 3; 1-4; 1, 2 and 5; 2 and 3; 2-4; 2 and 5; and 2-5, any of which may be in further combination with element(s), 9; 9 and 10; 9 and 11; 9-11; 12; 13; 14; 12 and 13; 12 and 14; 13 and 14; 12-14; 15; 16; 9 and 16; 9, 10 and 16; 9, 10 and 16; 9, 11 and 16; 9-11 and 16; 12 and 16; 13 and 16; 14 and 16; 15 and 16; 12, 13 and 16; 13, 14 and 16; 12-14 and 16; 15 and 16; 17; 9 and 17; 9, 10 and 17; 9, 11 and 17; 9-11 and 17; 12 and 17; 13 and 17; 14 and 17; 15 and 17; 12, 13 and 17; 12, 14 and 17; 13, 14 and 17; 12-14 and 17; 15 and 17; and 16 and 17. The method of B in combination with elements 9 and 10; 9 and 11; 9-11; 12 and 13; 12 and 14; 13 and 14; 12-14; 9 and 16; 9, 10 and 16; 9, 10 and 16; 9, 11 and 16; 9-11 and 16; 12 and 16; 13 and 16; 14 and 16; 15 and 16; 12, 13 and 16; 13, 14 and 16; 12-14 and 16; 15 and 16; 9 and 17; 9, 10 and 17; 9, 11 and 17; 9-11 and 17; 12 and 17; 13 and 17; 14 and 17; 15 and 17; 12, 13 and 17; 12, 14 and 17; 13, 14 and 17; 12-14 and 17; 15 and 17; and 16 and 17.

To facilitate a better understanding of the embodiments described herein, the following examples of various representative embodiments are given. In no way should the following examples be read to limit, or to define, the scope of the invention.

### EXAMPLES

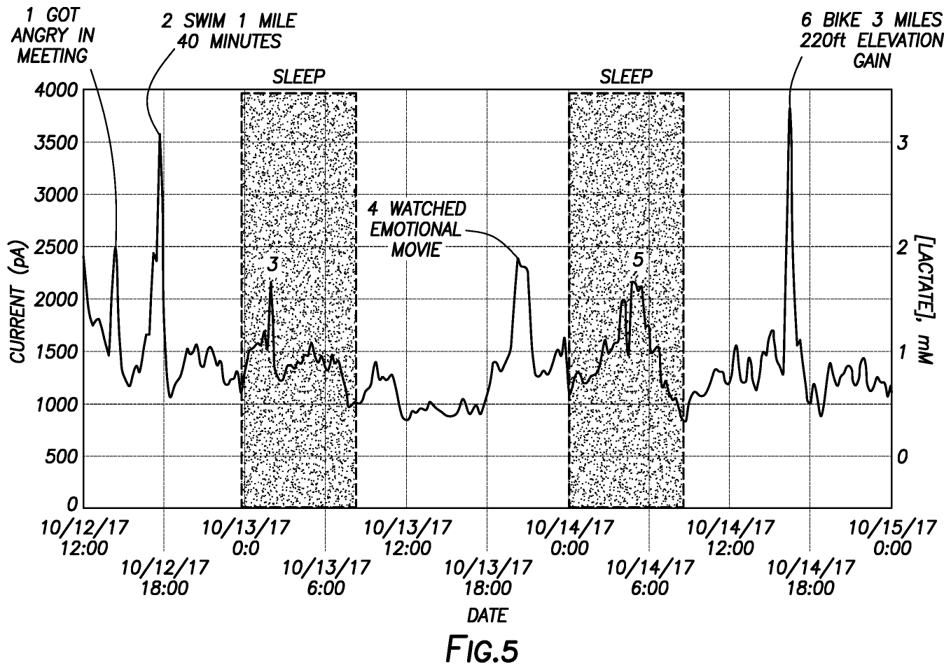

A FREESTYLE LIBRE™ (Abbott Diabetes Care) sensor housing was fitted with a lactate-responsive sensor, and lactate levels were monitored continuously in vivo for several days. The time was recorded at which each lactate concentration measurement was made. Concurrently, the time of day and physical activity were monitored using a clock and an exercise monitoring device, respectively. A plot of in vivo measured lactate concentrations as a function of time is shown in FIG. 5. Lactate concentrations above 1.5 mM were further analyzed as potentially being related to an emotional stress event.

As shown in FIG. 5, lactate spikes 3 and 5 were determined as being unrelated to emotional stress, since they occurred during confirmed non-waking hours. Similarly, lactate spikes 2 and 6 occurred in conjunction with exercise and were similarly determined to be unrelated to an emotional stress event. Lactate spikes 1 and 4, in contrast, occurred during waking hours and did not occur in conjunction with exercise. Provided that lactate spikes 1 and 4 do not occur as a result of eating, they may be correlated to an emotional stress event. Some individuals do not exhibit a lactate spike in conjunction with eating, whereas others do. As lactate spikes 1 and 4 occur, a wearer of the lactate-responsive sensor may be prompted to take further action.

Unless otherwise indicated, all numbers expressing quantities and the like in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

One or more illustrative embodiments incorporating various features are presented herein. Not all features of a physical implementation are described or shown in this application for the sake of clarity. It is understood that in the development of a physical embodiment incorporating the embodiments of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While various systems, tools and methods are described herein in terms of “comprising” various components or steps, the systems, tools and methods can also “consist essentially of” or “consist of” the various components and steps.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Therefore, the disclosed systems, tools and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems, tools and methods illustratively disclosed herein may suitably be practiced in the absence of any element that is not specifically disclosed herein and/or any optional element disclosed herein. While systems, tools and methods are described in terms of “comprising,” “containing,” or “including” various components or steps, the systems, tools and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

What is claimed is the following:

1. A sensing system comprising:
a lactate-responsive sensor adapted for detecting lactate in vivo; and
a processor communicatively coupled to the lactate-responsive sensor;
wherein the processor is adapted to determine a plurality of lactate concentrations measured by the lactate-responsive sensor over a period of time, and
wherein the processor is further adapted to correlate a lactate concentration spike, a lactate concentration change, a lactate concentration rate of change, or any combination thereof to an emotional stress event occurring within the period of time.

2. The sensing system of claim 1, wherein a housing for the lactate-responsive sensor is adapted to be worn on-body, and at least a portion of the lactate-responsive sensor protrudes from the housing for insertion in vivo.

3. The sensing system of claim 1, wherein the lactate-responsive sensor comprises a working electrode having a sensing layer comprising a lactate-responsive enzyme disposed thereupon.

4. The sensing system of claim 3, wherein the lactate-responsive enzyme is covalently bonded to a polymer comprising the sensing layer.

5. The sensing system of claim 3, wherein the sensing layer further comprises catalase as a stabilizer for the lactate-responsive enzyme.

6. The sensing system of claim 3, wherein the lactate-responsive enzyme is lactate dehydrogenase or lactate oxidase.

7. The sensing system of claim 1, further comprising:
a clock, an actimeter, an exercise monitor, or any combination thereof communicatively coupled to the processor;
wherein the processor is further adapted to determine, based upon a reading from the clock, the actimeter, the exercise monitor, or any combination thereof, that the lactate concentration spike, the lactate concentration change, the lactate concentration rate of change, or any combination thereof is not associated with an emotional stress event occurring within the period of time.

8. The sensing system of claim 1, further comprising:
a clock and an exercise monitor communicatively coupled to the processor;
wherein the processor is further adapted to determine, based upon a reading from the clock and a reading from the exercise monitor, that the lactate concentration spike, the lactate concentration change, the lactate concentration rate of change, or any combination thereof is not associated with an emotional stress event occurring within the period of time.

9. The sensing system of claim 1, further comprising:
a user input interface communicatively coupled with the processor;
wherein the user input interface is adapted for entering times associated with one or more of exercising, eating or sleeping.

10. A method comprising:
assaying a biological fluid in vivo with a lactate-responsive sensor adapted for measuring lactate in the biological fluid over a period of time;
communicating a signal from the lactate-responsive sensor to a processor;
determining a plurality of lactate concentrations with the processor using the signal communicated from the lactate-responsive sensor;
correlating, with the processor, a lactate concentration spike, a lactate concentration change, a lactate concentration rate of change, or any combination thereof to an emotional stress event occurring within the period of time; and
communicating a notification from the processor to an output medium that an emotional stress event has occurred.

11. The method of claim 10, wherein the lactate-responsive sensor comprises a working electrode having a sensing layer comprising a lactate-responsive enzyme disposed thereupon.

12. The method of claim 11, wherein the lactate-responsive enzyme is covalently bonded to a polymer comprising the sensing layer.

13. The method of claim 11, wherein the sensing layer further comprises catalase as a stabilizer for the lactate-responsive enzyme.

14. The method of claim 11, wherein the lactate-responsive enzyme is lactate dehydrogenase or lactate oxidase.

15. The method of claim 10, further comprising:
communicating one or more variables to the processor within the period of time, the one or more variables being selected from the group consisting of time of day, an actimetry reading, acceleration, position, altitude, heart rate, and any combination thereof; and
determining with the processor based upon the one or more variables whether the lactate concentration spike, the lactate concentration change, the lactate concentration rate of change, or any combination thereof is associated with an emotional stress event.

16. The method of claim 15, wherein, if the one or more variables are measured at a time of day specified for sleeping, the lactate concentration spike, the lactate concentration change, the lactate concentration rate of change, or any combination thereof is not communicated to the output medium as an emotional stress event.

17. The method of claim 15, wherein, if the one or more variables are determined by the processor to be suggestive of exercise, the lactate concentration spike, the lactate concentration change, the lactate concentration rate of change, or any combination thereof is not communicated to the output medium as an emotional stress event.

18. The method of claim 10, further comprising:
determining with the processor whether a user of the lactate-responsive sensor has been sleeping;
wherein, if the processor determines the user has been sleeping, the lactate concentration spike, the lactate concentration change, the lactate concentration rate of change, or any combination thereof is not communicated to the output medium as an emotional stress event.

19. The method of claim 10, further comprising:
determining with the processor whether a user of the lactate-responsive sensor has been exercising;
wherein, if the processor determines the user has been exercising, the lactate concentration spike, the lactate concentration change, the lactate concentration rate of change, or any combination thereof is not communicated to the output medium as an emotional stress event.

20. The method of claim 10, further comprising:
determining with the processor whether a user of the lactate-responsive sensor has been eating;
wherein, if the processor determines the user has been eating, the lactate concentration spike, the lactate concentration change, the lactate concentration rate of change, or any combination thereof is not communicated to the output medium as an emotional stress event.

21. The method of claim 10, further comprising:
determining with the processor whether a user of the lactate-responsive sensor has been sleeping, exercising, eating, or any combination thereof;
wherein, if the processor determines the user has been sleeping, exercising, eating, or any combination thereof, the lactate concentration spike, the lactate concentration change, the lactate concentration rate of change, or any combination thereof is not communicated to the output medium as an emotional stress event.

22. The method of claim 10, further comprising:
prompting a user of the lactate-responsive sensor for a response after communicating the notification of the emotional stress event to the output medium.

23. The method of claim 10, further comprising:
receiving one or more user inputs with the processor, the one or more user inputs comprising a range of times associated with one or more of exercising, eating, sleeping or any combination thereof; and
determining with the processor based upon the one or more user inputs whether the lactate concentration spike, the lactate concentration change, the lactate concentration rate of change, or any combination thereof is associated with an emotional stress event.
